(19) United States
(12) Patent Application Publication
(10) Pub. No.: US 2015/0320584 A1
(43) Pub. Date: Nov. 12, 2015

(54) OSTOMY SILENCER AND METHOD

(71) Applicant: Mayo Foundation for Medical Education and Research, Rochester, MN (US)

(72) Inventors: Michael E. Kralovec, Rochester, MN (US); Ann L. Leland, Pine Island, MN (US); Stephanie A. Beer, Burlington, VT (US); Michael B. Larson, Red Wing, MN (US)

(21) Appl. No.: 14/423,820

(22) PCT Filed: Aug. 30, 2013
(86) PCT No.: PCT/US2013/057562

§ 371 Date: Feb. 25, 2015

Related U.S. Application Data
(60) Provisional application No. 61/695,024, filed on Aug. 30, 2012.

(51) Int. Cl. A61F 5/44; G10K 11/178; A61F 5/445

(52) U.S. Cl. CPC A61F 5/4404; A61F 5/445; G10K 11/1782

(57) ABSTRACT

Devices and methods for reducing undesirable noises relating to ostomy bag usage. One embodiment is a sleeve of foam or other sound absorbing material configured to surround substantially all or part of an ostomy bag. The sleeve can have a zippered bag opening to releasably receive the ostomy bag, and a stoma-coupling opening to enable a patient's stoma to be coupled to the ostomy bag. Another embodiment of the sleeve includes a pocket and an active noise cancellation device held in the pocket.

### FIELD OF THE INVENTION

The present invention relates generally to devices and methods for reducing the volume of sound emanating from a stoma, an artificial hole in an abdomen, in patients such as those that have undergone a colostomy, ileostomy or jejunostomy.

### BACKGROUND

Bowel-related diseases and other medical issues are sometimes treated through surgical procedures such as colostomy, ileostomy or jejunostomy. During these surgical procedures the patient's bowel is redirected out of his or her abdomen through a hole known as a stoma. Body waste such as stool, flatus or gas and other fluids exit through this hole rather than the rectum. The stoma does not have a sphincter under voluntary muscular control by the patient. Because the patient's intestinal motion is an involuntary action, contractions that cause the expulsion of stool and gas are not able to be suppressed by the patient. A sealed waste collection device, commonly referred to as an ostomy bag, is therefore placed around and attached to the stoma to collect the waste. The bag is typically attached to the patient's skin around the stoma with tape or glue sealant, and is periodically changed.

Patients are usually able to put the bag under their existing clothing to cover and prevent the visual appearance of the bag. Unfortunately, the inability to control noises or sounds made when waste such as stool and gas exits the stoma unexpectedly presents continuing problems. For example, some patients report not wanting to engage in social interactions or to attend routine social events such as religious services and family gatherings because of embarrassment caused by these noises at inopportune times. These situations can negatively affect the quality of life, and sometimes even cause significant emotional distress, in patients that might otherwise be able to enjoy a fulfilling life free from other complications associated with the surgery.

Devices that function as patient-controlled valves to manually block the stoma are known. These devices can be actuated or otherwise controlled by the patient to release the waste at times selected by the patient. One such device, known as the Comfort-63, is commercially available from Stomabags of Miami, Fla.

There remains, however, a continuing need for devices and methods that alleviate certain complications such as those described above associated with the use of ostomy bags.

### SUMMARY

Embodiments of the invention include devices and methods to reduce undesirable noise-related complications related to the use of ostomy bags. One embodiment of the invention includes a sleeve of sound absorbing material configured to surround substantially all or part of an ostomy bag. Some embodiments include a bag opening that is closable by a zipper to removably receive the ostomy bag. Other embodiments include a stoma-coupling opening to enable a patient's stoma to be coupled to the ostomy bag. Yet other embodiments include a pocket for releasably holding an active noise cancellation device.

### BRIEF DESCRIPTION OF THE DRAWINGS

FIG. 1A is a plan view of a back or patient-facing side of an ostomy silencer in accordance with one embodiment of the present invention.

FIG. 1B is a plan view of an opposite, front side of the ostomy silencer shown in FIG. 1.

FIG. 2 is a block diagram of an active noise cancellation device that can be used with the ostomy silencer shown in FIGS. 1A and 1B.

FIG. 3A is an isometric view of the bottom or inside of an ostomy bag cap that includes an active noise cancellation device in accordance with another embodiment of the invention.

FIG. 3B is a top plan view of the ostomy bag cap shown in FIG. 3A.

FIG. 4 is a plan view of the back side of an ostomy bag that can be used with embodiments of the invention.

FIG. 5 is a detailed view of the bags as shown in FIG. 4, illustrating the removal of the baseplate from the collection portion.

FIG. 6 is a diagrammatic illustration of an active ostomy silencer in accordance with an embodiment of the invention.

FIG. 7 is an illustration of a patient wearing an ostomy bag of the type shown in FIGS. 4 and 5 with an ostomy silencer of the type shown in FIG. 6 mounted thereto.

FIG. 8 is an illustration of an active ostomy silencer in accordance with another embodiment of the invention in relation to an ostomy bag with which the silencer can be used.

FIG. 9 is an illustration of the frequency pass band of an embodiment of the sound cancellation electronics shown in FIG. 2

### DESCRIPTION OF THE INVENTION

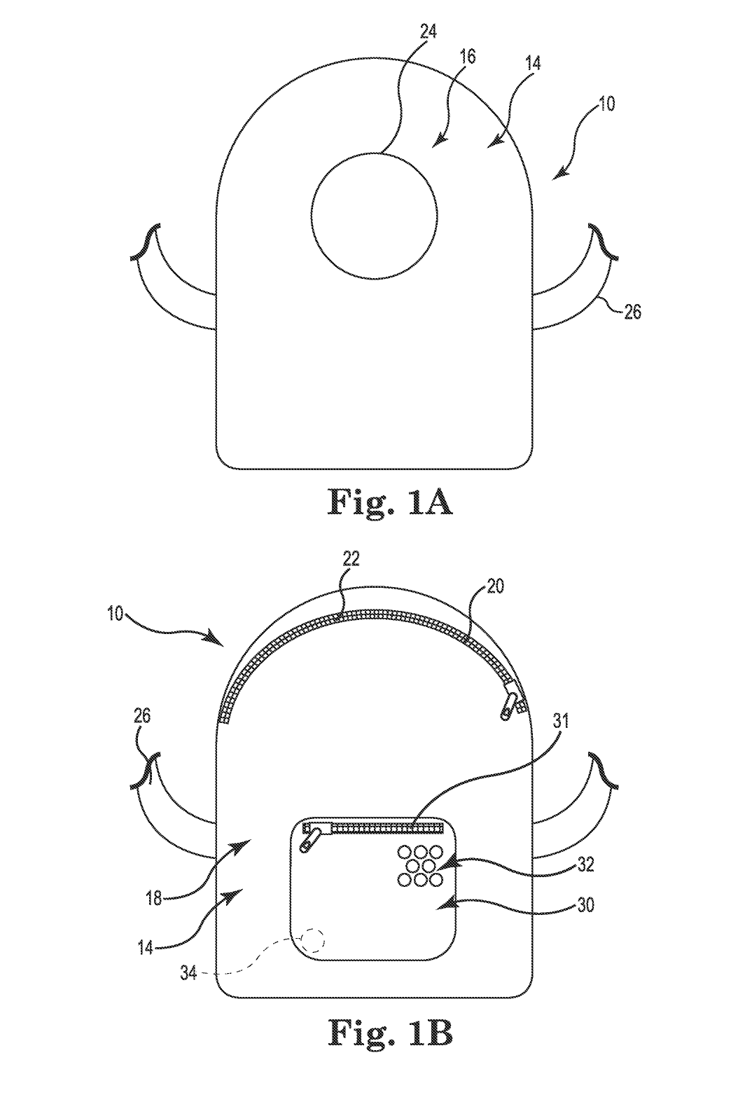

An ostomy silencer 10 in accordance with one embodiment of the invention is illustrated in FIGS. 1A and 1B. One embodiment of an active noise cancellation device 12 that is in some embodiments of the invention used in connection with the ostomy silencer 10 is shown in FIG. 2. Silencer 10 includes a sleeve 14 of sound absorbing or deadening material that is sized and shaped to surround substantially all or most of the patient's ostomy bag (not shown). Many commercially available ostomy bags come in standard sizes, and the sleeve 14 can be shaped and sized to fit those standard sized ostomy bags. In the illustrated embodiment, the sleeve 14 is formed as a generally closed bag or pouch having a first or patient-facing back side 16, a second or front side 18, and an opening 20 through which the ostomy bag can inserted into the sleeve 14. A zipper 22 is attached to the sleeve 14 to enable the opening 20 to be opened and closed or sealed. Other embodiments of the sleeve 14 have other structures (not shown) to close the opening 20. Still other embodiments (not shown) do not have structures for closing the opening. The sleeve 14 can, for example, be constructed from foam materials such as high density neoprene, open cell rubber foams, melamine sponges, Helmholtz resonators, honeycomb air chambers, viscoelastic-rubber backed material, and the like. The material of the sleeve 14 can, but need not be water-repellant or waterproof, and can have an optional anti-microbial coating. An opening 24 through the sleeve 14 (on the patient-facing side 16 in the illustrated embodiment) enables the opening in the ostomy bag (not shown) to be coupled to the patient's stoma. The illustrated embodiment of silencer 10 also includes an optional belt or strap 26 that can be used to releasably secure the silencer to the patient. Strap 26 can, for example, be worn around the patient's waist. Strap 26 can be removably mounted to the sleeve 14. Because of the sound absorbing properties of the sleeve 14, sounds associated with waste exiting the stoma are reduced.

The embodiment of the ostomy silencer 10 shown in FIGS. 1A and 1B is configured to be used with the active noise cancellation device 12 shown in FIG. 2. In this embodiment, the noise cancellation device 12 is removably held in a pocket 30 in or on the sleeve 14. Pocket 30 is sized to receive the noise cancellation device 12, and in the illustrated embodiment is located on the front side 18 of the sleeve 14. In other embodiments of the invention, a noise cancellation device such as 12 can be attached directly to the ostomy bag, or integrated into or mounted to a cap on the ostomy bag. The pocket 30 optionally includes a structure such as zipper 31 to seal the pocket and help retain the noise cancellation device 12 therein. To enhance the functionality and effects of the microphone and speaker of the noise cancellation device 12 (described below), the illustrated embodiment of the pocket 30 includes a speaker opening 32 extending from the front side 18 of the sleeve 14, and a microphone opening 34 that faces the interior of the sleeve and is located next to a colostomy bag within the sleeve.

Noise cancellation device 12 includes an enclosure 50 containing a battery compartment 52, sound cancellation electronics 54, speaker 56 and microphone 58. Speaker 56 can be mounted to face a first side of enclosure 12, and positioned on the enclosure at a location causing it to be positioned adjacent to the speaker opening 32 on the pocket 30. Speaker 56 is thereby configured to effectively broadcast outwardly from the pocket 30 and away from the patient. Microphone (mic) 58 is mounted to face a second, opposite side of the enclosure 50 in the illustrated embodiment, and is positioned on the enclosure at a location causing it to be positioned adjacent to the microphone opening 34 in the pocket 30. The microphone 58 is thereby configured to efficiently detect sounds emanating from the stoma (i.e., and present in the ostomy bag). Battery compartment 53 can be enclosed by a cover 60. Sound cancellation electronics 54 can include threshold detector 62. As shown in FIG. 2, the sound cancellation electronics 54 is coupled to and powered by a battery (not shown) in the compartment 52. Speaker 56 and microphone 58 are also connected to the sound cancellation electronics 54.

Noise cancellation device 12 is an active device that cancels noise emanating from the stoma. The device 12 uses known approaches to provide this result. Briefly, microphone 58 detects sounds emanating from the stoma and couples signals representative of those sounds to the sound cancellation electronics 54. Sound cancellation electronics 54 produce an output signal having a frequency content that is generally the same as that of the signal from the microphone, but one hundred and eighty degrees out of phase with the microphone signal. The speaker 56 thereby produces a sound similar to, but one hundred and eighty degrees out of phase with, the sound detected by the microphone 58. The sound cancellation electronics also causes the sound produced by the speaker 56 to have an amplitude or volume similar to that of the sound detected by the microphone 58. Automatic or other volume control methodologies can provide this function. Because the sound waves produced by the speaker 56 are generally the same as those emanating from the stoma, but of opposite phase, the sound waves produced and broadcast by the speaker effectively cancel the sound waves emanating from the stoma. This effect of the noise cancellation device 12 is enhanced because the speaker 56 is located close to the source of the undesired sounds. Sounds associated with stool, fluids and gas exiting the stoma are thereby substantially reduced in volume or effectively eliminated. Although shown and described in connection with the passive sound reduction functionality produced by the sleeve 14, noise cancellation device 12 is used independently of the sleeve in other embodiments of the invention. For example, the noise cancellation device 12 can be functionally coupled to the patient and his or her ostomy bag by other structures or approaches.

Sound cancellation electronics 54 can be implemented in any of a wide variety of known or otherwise conventional approaches such as a programmed microprocessor, an ASIC (application specific integrated circuit) or using discrete circuit components. The illustrated embodiment of sound cancellation electronics 54 includes threshold detector 62 that causes the electronics to generate an output signal to the speaker 56 only when the detected sounds reach or exceed a certain threshold (e.g., volume) level. The threshold level can be selected by the patient in some embodiments of the invention. By not responding to relatively low level stoma-related sounds that might not be sufficiently audible to be of concern to the patient, more efficient battery use can be achieved. Other embodiments of the invention do not include the threshold detector 62.

Any suitable noise cancellation circuit may be utilized in device 12. By way of example, embodiments of the device can include a circuit incorporating a microphone, a signal inverting amplifier, and a speaker, where said signal inverting amplifier inverts the phase of the signal from the microphone which is then output to a speaker in such a manner as to attenuate the sound from the original source with destructive wave interference. The circuit may also be modified with various filters, be limited to lower frequencies, include an amplifier on the output, incorporate the use of multiple microphones at different locations, incorporate a control for the separation between the noise source and cancellation speaker, include adaptable microprocessors with algorithms designed to actively adjust the speaker output, and the like. These modifications may have advantages with increasing the effective range, expanding the power band, and allow for cancellation of reverberations or echoes. Noise cancellation devices are disclosed, for example, in the Lueg U.S. Pat. No. 2,043,416, the Bergeron U.S. Pat. No. 8,189,803 and European Patent Application Publication No. 0 040 462, all of which are incorporated herein by reference in their entirety and for all purposes.

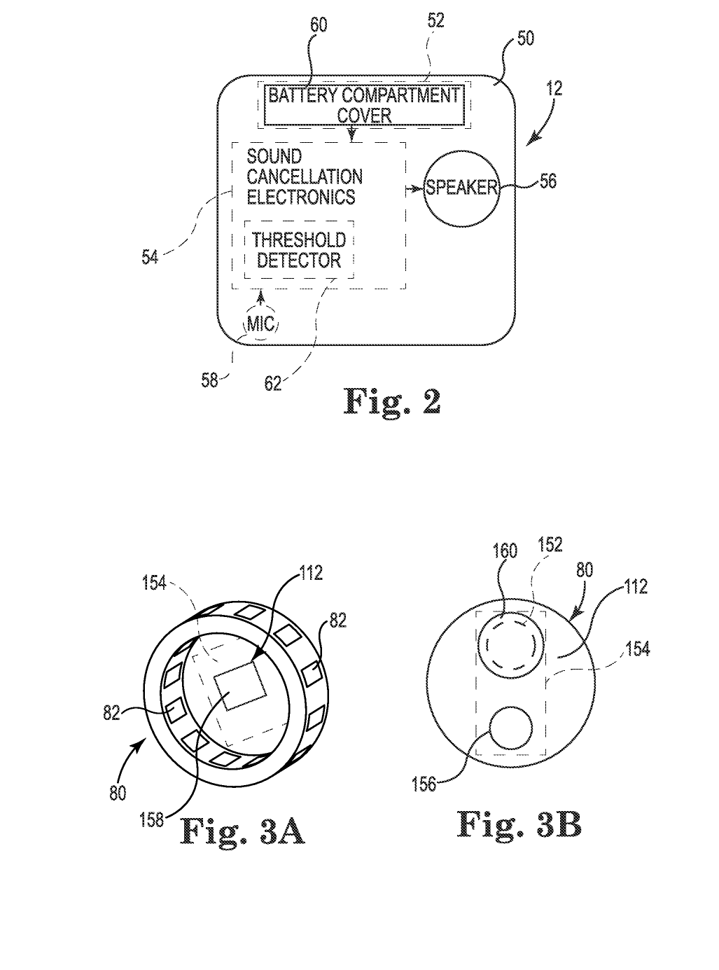

FIGS. 3A and 3B illustrate bottom and top views, respectively, of an ostomy bag cap 80 having a noise cancellation device 112 incorporated into the cap. Noise cancellation device 112 can be similar to noise cancellation device 12 described above, and similar elements are identified by similar reference numbers. In the illustrated embodiment a battery compartment cover and speaker are mounted to the top or outer surface of the cap 80, and a microphone 158 is mounted to the inside surface of the cap. A liquid impermeable membrane (not visible) can cover the microphone 158. Sound cancellation electronics 154 can be embedded within or mounted to the cap 80. Gas openings 82 extend through the side wall of the cap 80.

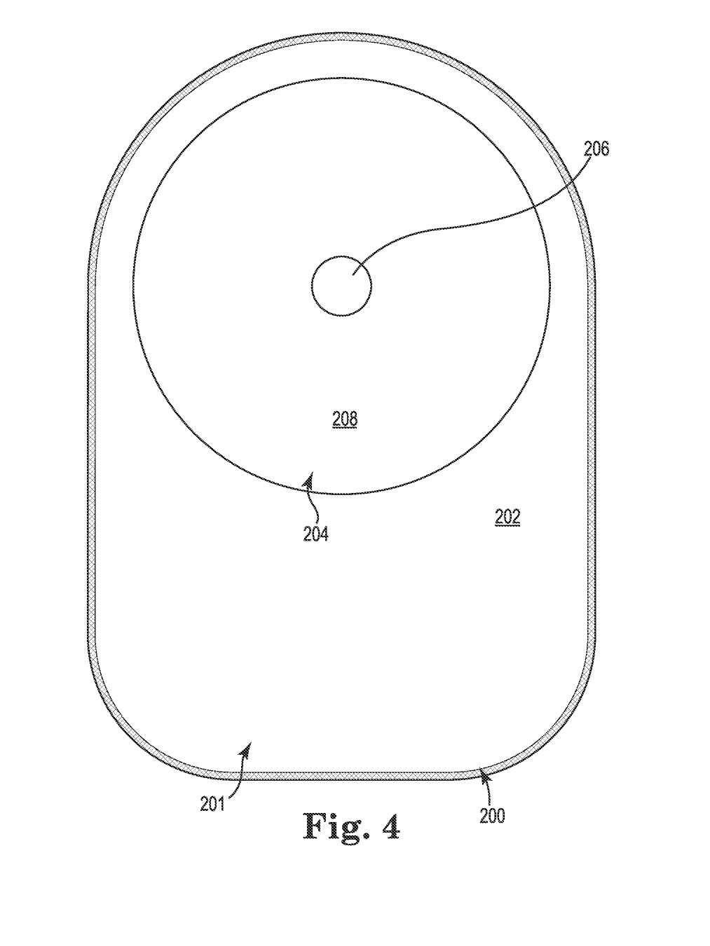

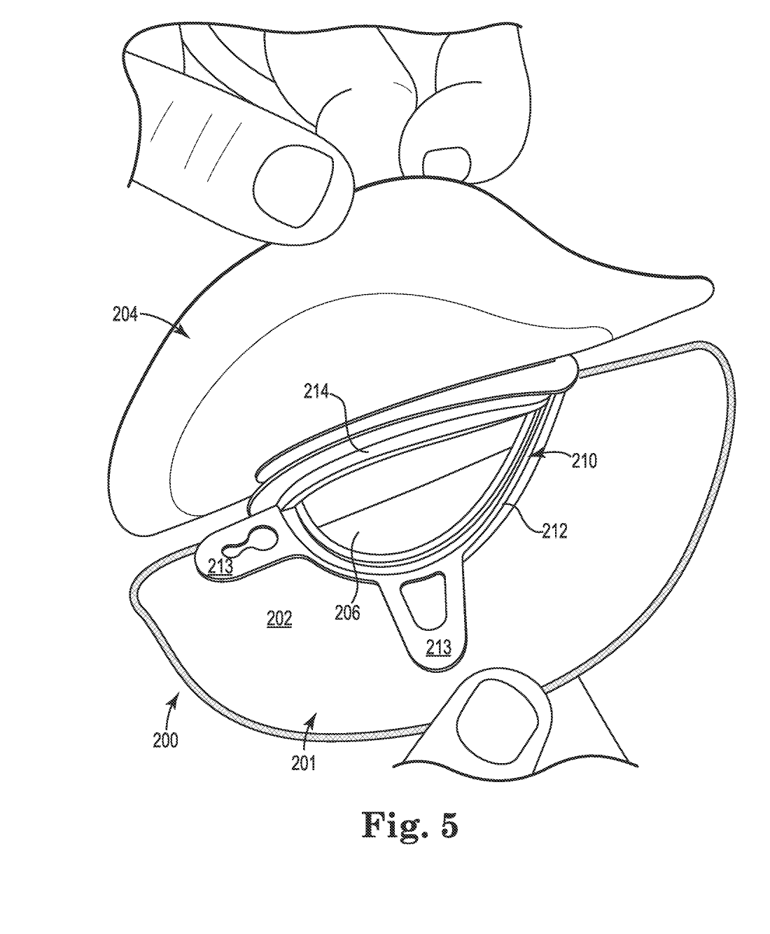

FIGS. 4 and 5 illustrate one example of an ostomy bag 200 with which embodiments of the invention can be used. Bag 200 includes a collection portion 201 having a front side (not visible in FIGS. 4 and 5), a back or patient-facing side 202, and a base plate 204 on the back side of the collection portion. As shown, a stoma opening 206 extends into the interior of the collection portion through the base plate 204 and the back side 202. A back or patient-facing side 208 of the base plate 204 includes an adhesive surface (optionally covered by a release liner prior to use) that can be used to attach the bag 200 to a patient's body with the stoma opening 206 aligned with the patient's stoma. In the illustrated embodiment, the collection portion 201 and base plate 204 are releasably and fluidly connected by a seal 210 having a first or collection portion seal member 212 and a second or base plate seal member 214. Seal members 212 and 214 can be press fit and released from one another by hand. In the illustrated embodiment the collection portion seal member 212 includes tabs 213. Ostomy bag 200 is shown for purposes of illustration only, and embodiments of the invention can be used with other ostomy bags. For example, other ostomy bags (not shown) may not include the base plate 204 or a releasable seal 210, and embodiments of the invention can be used with these and other types of ostomy bags.

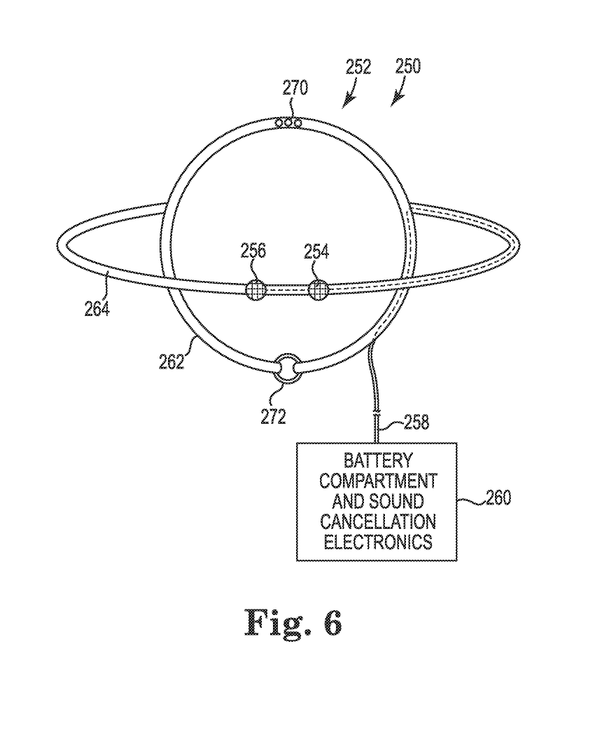

FIG. 6 is an illustration of an ostomy silencer 250 in accordance with another embodiment of the invention. The illustrated embodiment of silencer 250 includes a support 252 that is configured to be mounted to an ostomy bag, and a microphone 254 and speaker 256 mounted to the support. A wire connector 258 electrically couples the microphone 254 and speaker 256 to an enclosure 260 that can include a battery compartment and sound cancellation electronics. The enclosure 260 and the battery compartment and sound cancellation electronics can, for example, be substantially the same as or similar to those of active noise cancellation device 12 described above in connection with FIG. 2. By coupling the support 252 and components thereon to the enclosure 260 by wire or other approaches (e.g., wireless communication approaches), the enclosure and components thereon can be attached to the patient's clothing (e.g., in a pocket) and conveniently and discretely worn. The physical size of the silencer 250 can also be minimized. In other embodiments, some or all of the components in the enclosure 260 can alternatively be mounted to the support 252. Similarly, components such as microphone 254 and speaker 256 can be located on the enclosure 260.

The illustrated embodiment of the support 252 includes an attachment portion 262 configured to extend around the stoma opening 206 on the back side 202 of the ostomy bag 200, and a mount portion 264 that extends from the attachment portion. One or both of the microphone 254 and speaker 256 are mounted to the mount portion 264. The mount portion 264 is configured to locate one or both of the microphone 254 and speaker 256 at a location that is not behind (i.e., is out from behind) the ostomy bag 200 when the bag and silencer 250 are worn by a patient. In some embodiments, the operation of the silencer 250 can be optimized when the mount portion 264 is configured to locate the microphone 254 and/or speaker 256 adjacent a front side of the ostomy bag 200, opposite the bag from the stoma, when the bag is worn by the patient.

Attachment portion 262 is a ring-shaped and annular member in the illustrated embodiment, although other embodiments take other shapes. The illustrated embodiment of the attachment portion 262 also includes a hinge 270 and clasp 272 that enables the attachment portion to be opened and closed to facilitate attachment to the ostomy bag 200. For example, the attachment portion 262 can be opened at the hinge 270, positioned around the seal 210 (e.g., between the base plate 204 and collection portion 201), and then closed with the clasp 272. Other embodiments of the attachment portion 262 have other structures to enable the attachment portion to be mounted to the ostomy bag 200 (e.g., the attachment portion 262 can be elastically expandable or formed of shape memory material). In still other embodiments the attachment portion 262 can be one piece or relatively rigid, and attached to the bag when the collection portion 201 is separated from the base plate 204 or by inserting the flexible collection portion through the attachment portion. Yet other embodiments of the invention have attachment portions that are not ring-shaped. For example, the attachment portion can be a clip that attaches to one of the tabs 213, on the ostomy bag 200.

The mount portion 264 is an elongated loop or handle in the embodiment illustrated in FIG. 6 to enable to mount portion to extend around the ostomy bag 200. In some embodiments the mount portion 264 can be attached to the attachment portion 262 by a hinge other flexible connection structure to enable the attachment portion to be conveniently moved over the ostomy bag 200 and to lay flat on the bag. Although the illustrated embodiment of the mount portion 264 is connected to the attachment portion 262 on its opposite sides at two locations, other embodiments the mount portion are attached to the attachment portion at one or more than two locations.

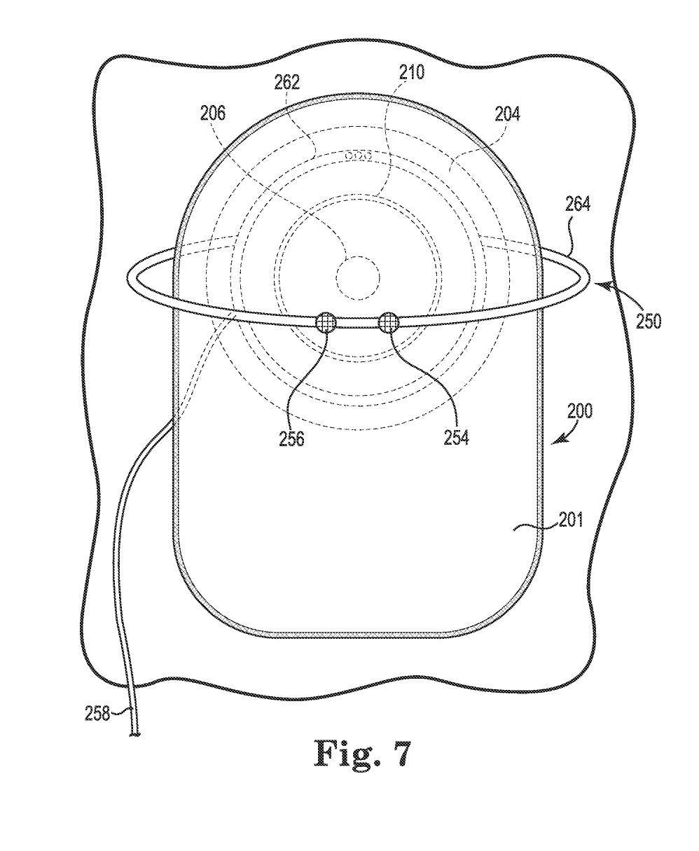

FIG. 7 is an illustration of an ostomy bag 200 and a silencer 250 of the type described above mounted to a patient.

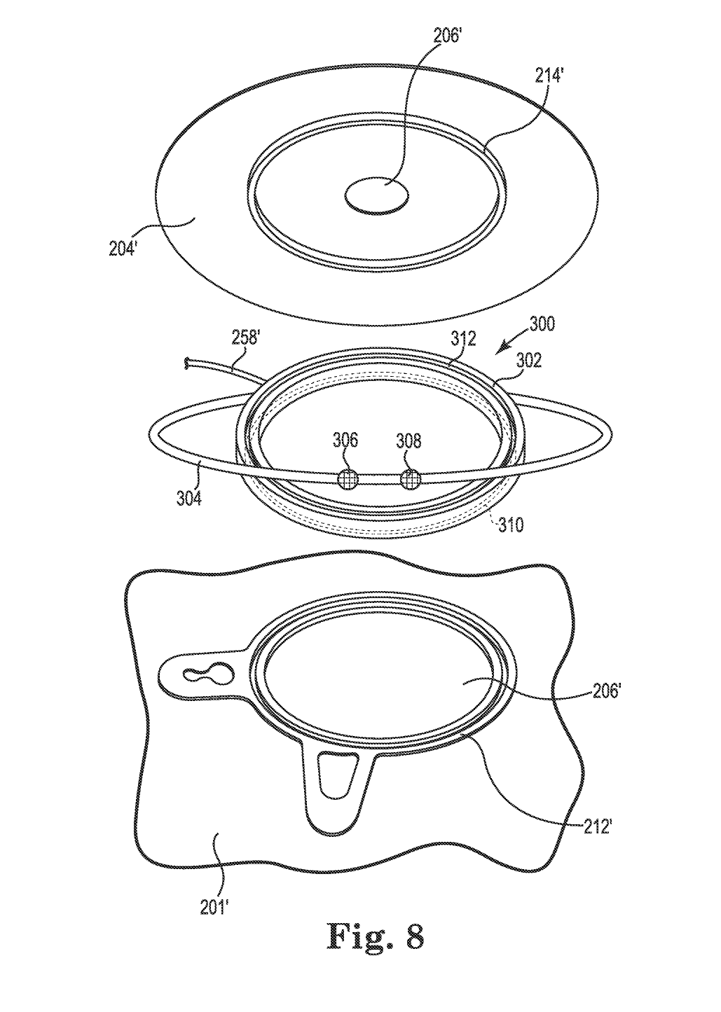

FIG. 8 is an illustration of an ostomy silencer 300 in accordance with another embodiment of the invention. As shown, silencer 300 is configured for use in connection with an ostomy bag 200′ having a collection portion 201′ with a first seal component 212′ and a base plate 204′ with a second seal component 214′. Ostomy silencer 300 releasably and fluidly couples the collection portion 201′ to the base plate 204′, and includes a body 302 having first and second opposite sides with an opening extending between the opposite sides. A mount portion 304 extends from the body 302 and carries one or both of a microphone 306 and speaker 308. A first coupling structure such as seal component 310 is located on the first side of the body 302 and is configured to releasably and fluidly couple the body to the stoma opening 206′ on the collection portion 201′. In the illustrated embodiment, for example, the seal component 310 releasably couples to the seal component 212′ on the collection portion 201′. A second coupling structure such as seal component 312 is located on the second side of the body 302 and is configured to releasably and fluidly couple the body to the opening in the ostomy bag base plate 204. In the illustrated embodiment, for example, the seal component 312 releasably couples to the seal component 214′ on the base plate 204′. Although not shown in FIG. 8, silencer 300, and in particular the microphone 306 and/or speaker 308 can be coupled (e.g., by a wire connecter 258′) to an enclosure such as 260 of the type described above having the battery compartment and noise cancellation electronics. In other embodiments one or more of the components in the enclosure 260 can be mounted to the body 302.

Embodiments of the invention such as silencers 250 and 300 can be made relatively thin and have a low profile to minimize their physical size, and their contribution to the physical appearance of the ostomy bag when worn by the patient. For example, in embodiments the attachment portion 262 of silencer 250 and the body 302 of the silencer 300 can be 1 cm or less in thickness. Other embodiments of silencers 250 and 300 have other sizes.

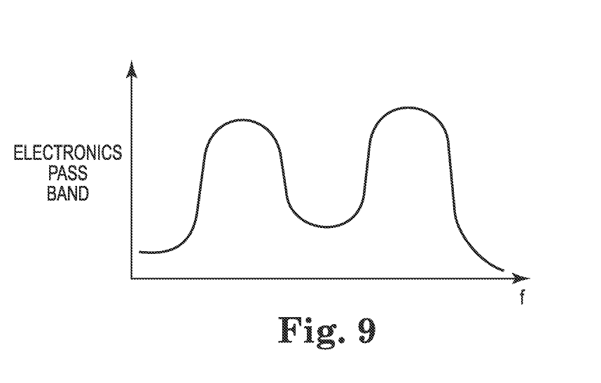

Sound emanating from a patient's stoma sometimes has predominant or substantial sound components in different frequency bands, and relatively quiet components in other frequency bands. The frequency bands of the substantial components are sometimes separated from one another by the frequency bands of the relatively quiet sounds. For example, the sounds of solids flowing into the ostomy bag can be formed predominantly or largely of frequencies in a first and relatively low band, and the sounds of liquid flowing into the ostomy bag can be formed predominantly and largely of frequencies in a second and relatively high band that is spaced from the solids frequency band. The sounds emanating from the stoma may have relatively little frequency content in the range between the solids and liquids frequency bands. In embodiments of the invention, the sound cancellation electronics such as 54 described in connection with FIG. 2 can be configured to have pass bands that correspond to the predominant solids and liquid frequencies. The processing capabilities of the sound cancellation electronics can thereby be optimized by being devoted to the frequency content that corresponds most directly with the undesired noises emanating from the stoma. FIG. 9 is an illustration of the pass band frequency characteristics of embodiments of the sound cancellation electronics having these features.

Embodiments of the invention offer a number of important advantages. Embodiments that include a sleeve are convenient and easy to use and maintain. It is lightweight and comfortable to wear. Importantly, it also substantially reduces problematic stoma-related noises. The noise cancellation device can be a small and thin device. Embodiments of the invention that include this device or method are convenient to use. For example, the device can be conveniently mounted to the sleeve, ostomy bag cap or other structures near the stoma. Because it can removably mounted to the sleeve (e.g., in the pocket) in some embodiments, the noise cancellation device can be easily cleaned if it becomes soiled. The silencer requires no control actions or operation by the patient or physician. There is no physical interaction with the stoma itself, so the device presents no potential for medical complications associated with bowel obstruction. Sterility can be maintained and opportunities for infection transmission are minimized because the device does not directly contact the ostomy, stool, gas or other body fluids. The device is essentially universal, in that it can be used with a wide variety of types of ostomies, ostomy bags and related equipment. For these and other reasons it can have a significant positive impact on the quality of life of stoma patients.

Although the invention has been described with reference to preferred embodiments, those skilled in the art will recognize that changes can be made in form and detail without departing from the spirit and scope of the invention.

1. An ostomy silencer including a sleeve of sound absorbing material configured to surround substantially all or part of an ostomy bag.

2. The ostomy silencer of claim 1 wherein the sleeve includes a stoma-coupling opening to enable a patient's stoma to be coupled to the ostomy bag.

3. The ostomy silencer of claim 2 wherein the sleeve includes a bag opening to removably receive the ostomy bag.

4. The ostomy silencer of claim 3 and wherein the bag opening is closable.

5. The ostomy silencer of claim 4 and further including a closure structure on the bag opening.

6. The ostomy silencer of claim 5 wherein the sleeve includes or is constructed substantially of foam material.

7. The ostomy silencer of claim 1 and further including an active noise cancellation device.

8. The ostomy silencer of claim 7 wherein the sleeve includes a device receiving structure, optionally including a pocket, for releasably holding the noise cancellation device.

9. The ostomy silencer of claim 8 wherein the device receiving structure includes an opening.

10. The ostomy silencer of claim 9 and further including a closure, optionally including a zipper, on the opening of the device receiving structure.

11. The ostomy silencer of claim 7 and further including a microphone opening through the sleeve, and wherein the microphone opening is adjacent to a microphone of the active noise cancellation device.

12. The ostomy silencer of claim 11 and further including a speaker opening, and wherein the speaker opening is adjacent to a speaker of the active noise cancellation device.

13. The ostomy silencer of claim 7 wherein the noise cancellation device includes:
sound cancellation electronics;
a speaker coupled to the sound cancellation electronics; and
a microphone coupled to the sound cancellation electronics.

14. A method for silencing stoma-related noise emanating from a surgery patient, including:
detecting stoma-related noise;
generating and broadcasting a general replica of the stoma-related noise that is about one hundred and eighty degrees out of phase with the detected noise.

15. An ostomy silencer, including:
an active noise cancellation device including:
sound cancellation electronics;
a speaker coupled to the sound cancellation electronics; and
a microphone coupled to the sound cancellation electronics; and
a support for holding the active noise cancellation device on a patient adjacent to the patient's stoma.

16. The ostomy silencer of claim 15 wherein the support holds the active noise cancellation device adjacent to a patient's ostomy bag.

17. The ostomy silencer of claim 15 wherein the support includes an ostomy bag cap.

18. The ostomy silencer of claim 17 wherein the active noise cancellation device is integrated into the cap.

19. An ostomy silencer, including:
a support, the support configured for attachment to an ostomy bag; and
one or both of a microphone and speaker mounted to the support, the one or both of the microphone and speaker configured to be coupled to sound cancellation electronics.

20. The ostomy silencer of claim 19 and further including sound cancellation electronics mounted to the support and coupled to the one or both of the microphone and speaker.

21. The ostomy silencer of claim 20 wherein the sound cancellation electronics includes a first and optionally solids sound frequency pass band and a second and optionally liquids sound frequency pass band that are separated from one another.

22. The ostomy silencer of claim 19 and further including:
sound cancellation electronics separate from the support and configured for attachment to a patient's clothing; and
means for coupling the sound cancellation electronics to the one or both of the speaker and microphone.

23. The ostomy silencer of claim 22 wherein the sound cancellation electronics includes a first and optionally solids sound frequency pass band and a second and optionally liquids sound frequency pass band that are separated from one another.

24. The ostomy silencer of claim 19 wherein the support is configured to locate one or both of the microphone and speaker out from behind the ostomy bag when the bag is worn by a patient.

25. The ostomy silencer of claim 24 wherein the support is configured to locate one or both of the microphone and speaker adjacent a front of the ostomy bag and opposite the bag from the stoma when the bag is worn by a patient.

26. The ostomy silencer of claim 19 wherein the support is configured for attachment to a seal component on an ostomy bag.

27. The ostomy silencer of claim 19 wherein the support includes:
an attachment portion configured to extend around a stoma opening on a back side of the ostomy bag; and
a mount portion extending from the attachment portion to a location out from behind the ostomy bag when the bag is worn by a patient, and wherein one or both of the microphone and speaker is mounted to the mount portion.

28. The ostomy silencer of claim 27 wherein the mount portion of the support is configured to locate one or both of the microphone and speaker adjacent a front of the ostomy bag and opposite the bag from the stoma when the bag is worn by a patient.

29. The ostomy silencer of claim 28 wherein the mount portion is movable with respect to the attachment portion.

30. The ostomy silencer of claim 27 wherein the attachment portion includes a ring member.

31. The ostomy silencer of claim 30 wherein the ring member is configured to be opened and closed, and optionally includes one or both of a hinge and a clasp.

32. The ostomy silencer of claim 19 wherein the support is configured to releasably and fluidly couple a stoma opening of the ostomy bag to a patient's stoma

33. The ostomy silencer of claim 32 wherein the support includes:
a body having first and second opposite sides and an opening extending between the first and second sides;
a first coupling structure on the first side of the body, the first coupling structure configured to be releasably and fluidly coupled to an opening on an ostomy bag base plate; and
a second coupling structure on the second side of the body, the second coupling structure configured to be releasably and fluidly coupled to the stoma opening of the ostomy bag.
